(19) United States
(12) Patent Application Publication
(10) Pub. No.: US 2012/0203249 A1
(43) Pub. Date: Aug. 9, 2012

(54) METHOD AND DEVICE FOR TENDON REPAIR

(75) Inventors: Christopher Charles Schmidt, Bridgeville, PA (US); David Michael Weir, II, West Homestead, PA (US)

(73) Assignee: Allegheny-Singer Research Institute, Pittsburgh, PA (US)

(21) Appl. No.: 13/369,878

(22) Filed: Feb. 9, 2012

Related U.S. Application Data
(60) Provisional application No. 61/441,117, filed on Feb. 9, 2011.
(60) Provisional application No. 61/471,968, filed on Apr. 5, 2011.

(51) Int. Cl. A61B 17/04

(52) U.S. Cl. 606/144

(57) ABSTRACT

Embodiments herein are directed to an anchor and an insertion tool. The anchor may be sized for near cortex anchor fixation or for fixation into the intramedullary canal of a bone. Embodiments also describe a cortical anchor and insertion tool assembly including an anchor having a tool receptor, a leading face, a central face, a rear face, and a suture spool to receive suture that is situated away from a center of a longitudinal axis of the anchor; and an insertion tool having a rod member having a distal end. A tip of the rod member may include a pivot structure capable of actively pivoting the anchor at the tool receptor. In some embodiments, the anchor may be guided into a hole in a bone by the insertion tool and pivoted at the tool receptor to fully or partially cover the hole.

### CROSS-REFERENCE TO RELATED APPLICATIONS

This application claims the benefit of and priority from U.S. Patent Application No. 61/441,117 filed Feb. 9, 2011, and U.S. Patent Application No. 61/471,968 filed Apr. 5, 2011, the contents of each of which are incorporated by reference herein in its entirety.

### BACKGROUND

This document relates to methods and devices for tendon repair. Current cortical button techniques for the reattachment of tendon to the diaphysis of a bone involve drilling a hole through both cortices of the bone. The button is then passed through this hole and flipped on the outside portion of the far cortex using suture. In other techniques, when attaching a tendon to the metaphysis or epiphysis regions of a bone, a hole is drilled through the near cortex and an anchor is screwed into the hole, with the threads of the anchor holding in cancellous bone rather than cortical bone.

It is desirable that a cortical anchor be as small as possible in order to minimize damage to the tissue or bone in which the button is anchored. It is also desirable that a cortical anchor be easily attached, hold the tissue firmly in place, and be easily removable without trauma. Furthermore, when using the anchor to attach a tissue to the metaphysis or epiphysis regions of a bone, it is desirable that the anchor be shaped such that it can be placed and anchored in the metaphysis or epiphysis regions of a bone without needing to drill holes through both cortices of the bone.

### SUMMARY

Embodiments herein may be directed to an anchor or an anchor and insertion tool assembly. Some embodiments may describe a cortical anchor including a suture spool to receive suture that may be situated away from a center of a longitudinal axis of the anchor by from about 0.1 mm to about 5 mm. In some embodiments, the anchor may further include a bottom leading face and a bottom central face that form an angle from about 5 to about 45 degrees. In some embodiments, the anchor may further include a bottom rear face that, together with the bottom central face, forms an angle from about 5 to about 45 degrees. In some embodiments, the anchor may further include a tool receptor configured to engage a pivot structure. In some embodiments, the tool receptor may be T-shaped, L-shaped, a ball socket, a slot, a hole, or a combination thereof. In some embodiments, the tool receptor may include curved walls to engage a pivot structure. In some embodiments, the anchor may be sized for near cortex anchor fixation in the metaphyseal or epiphyseal regions of bone. In some embodiments, the anchor may be sized for near cortex fixation inside the intramedullary canal of the diaphyseal region of a bone. In some embodiments, the anchor may be any size suitable for implantation in a bone, including, without limitation, bones of the shoulder, elbow, hand, wrist, knee or foot.

Embodiments herein also describe a cortical anchor and insertion tool assembly including an anchor having a tool receptor that receives a pivot structure; an insertion tool having a rod member having a distal end and a proximal end; and a tip at the distal end of the rod member including the pivot structure which is capable of actively pivoting the anchor at the tool receptor. In some embodiments, the tip may allow the pivot structure to be positioned in space or positioned beyond a desired fixation point before rotation.

In some embodiments, the anchor may be sized for near cortex anchor fixation in the metaphyseal or epiphyseal regions of bone. In some embodiments, the anchor may be sized for near cortex fixation inside the intramedullary canal of the diaphyseal region of a bone. In some embodiments, the anchor may be any size suitable for implantation in a bone, including, without limitation, bones of the shoulder, elbow, hand, wrist, knee or foot. In some embodiments, the tool receptor may include curved walls to engage the tip. In some embodiments, the anchor may have a suture spool to receive suture. In some embodiments, the assembly may further include a bottom leading edge having an angle from about 5 to about 45 degrees from a bottom edge of the anchor. In some embodiments, the anchor may further include a rear face and a central face that form an angle from about 5 to about 45 degrees. In some embodiments, the insertion tool may further include a cannulated covering which at least partially covers the rod member. In some embodiments, the cannulated covering may further include a lip to prevent the anchor from pivoting. In some embodiments, the insertion tool may further include an actuator to extend the rod member from the cannulated covering. In some embodiments, the tool receptor may be complementary to the pivot structure. In some embodiments, the suture spool may situated away from a center of a longitudinal axis of the anchor by from about 0.1 mm to about 5 mm.

Embodiments herein also describe a cortical anchor and insertion tool assembly including an anchor having a tool receptor, a leading face and a central face that form an angle from about 5 to about 45 degrees, a rear face that forms an angle from about 5 to about 45 degrees with the central face, and a suture spool; and an insertion tool having a rod member having a distal end and a proximal end, and a tip at the distal end of the rod member having a pivot structure capable of actively pivoting the anchor at the tool receptor. In some embodiments, the tip may allow the pivot structure to be positioned in space or positioned beyond a desired fixation point before rotation. In some embodiments, the anchor is capable of being guided into a hole in a bone by the insertion tool and pivoted at the tool receptor to cover the hole. In some embodiments, the suture spool may be situated away from a center of a longitudinal axis of the anchor by from about 0.1 mm to about 5 mm.

### DESCRIPTION OF DRAWINGS

For a fuller understanding of the nature and advantages of the present invention, reference should be had to the following detailed description taken in connection with the accompanying drawings, in which:

FIG. 1 illustrates an isometric top view of an anchor according to an embodiment described herein.

FIG. 2 illustrates an isometric back view of an anchor according to an embodiment described herein.

FIG. 3 illustrates a top view of an anchor according to an embodiment described herein.

FIG. 4 illustrates a side view of an anchor according to an embodiment described herein.

FIG. 5 illustrates a back view of an anchor according to an embodiment described herein.

FIG. 6 illustrates an isometric view of a portion of an inserter tool according to an embodiment herein.

FIG. 7 illustrates an isometric view of a portion of an inserter tool with a tapered rod member according to an embodiment herein.

FIG. 8 illustrates an isometric view of anchor designed to fit a tapered rod member according to an embodiment herein.

FIG. 9 illustrates an inserter tool according to an embodiment herein.

FIG. 10 illustrates an isometric top view of a retracted inserter tool according to an embodiment described herein.

FIG. 11 illustrates an isometric back view of a retracted inserter tool according to an embodiment described herein.

FIG. 12 illustrates isometric top view of a released inserter tool according to an embodiment described herein.

FIG. 13 illustrates an isometric back view of a released inserter tool according to an embodiment described herein.

FIG. 14 illustrates an isometric view of an anchor and tool assembly according to an embodiment described herein in which the anchor has been flipped.

FIG. 15 illustrates a side view of an inserter tool and anchor assembly according to an embodiment described herein where the button is in a retracted position.

FIG. 16 illustrates a side view of an inserter tool and anchor assembly according to an embodiment described herein where the button is in a released position, allowing the button to flip.

FIG. 17 illustrates a comparison of the structure of an anchor with a generally rectangular shaped cross-section; and an anchor according to an embodiment described herein.

FIG. 18 illustrates the angle of entry into the intramedullary canal of an anchor with a generally rectangular shaped cross-section.

FIG. 19 illustrates the angle of entry into the intramedullary canal of an anchor according to an embodiment described herein.

FIG. 20 illustrates a comparison of the angle of entry into the intramedullary canal of an anchor with a generally rectangular shaped cross-section; and an anchor according to an embodiment described herein.

FIG. 21 illustrates (A) a frontal view of a double row rotator cuff repair using anchors of embodiments described herein flipped within the cancellous region of bone in the humerus; (B) a lateral view of the double row rotator cuff repair of FIG. 18(A); and (C) a distal biceps tendon repair using an anchor of embodiments described herein flipped within the intramedullary canal of the radius.

### DETAILED DESCRIPTION

This invention is not limited to the particular processes, compositions, or methodologies described, as these may vary. The terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. All publications mentioned herein are incorporated by reference in their entirety. Nothing herein is to be construed as an admission that the invention is not entitled to antedate such disclosure by virtue of prior invention.

As used herein, the singular forms “a”, “an”, and “the” include plural reference unless the context clearly dictates otherwise. Thus, for example, reference to a “cortical button” is a reference to one or more cortical buttons and equivalents thereof known to those skilled in the art, and so forth.

As used in this document, the term “about” means plus or minus 10% of the numerical value of the number with which it is being used. Therefore, about 50% means in the range of 45%-55%.

As used in this document, the term “comprises” means includes at least the following but does not exclude others.

As used herein, the term “angle” means the space between two rays diverging from a common end point or vertex. The rays may be curved or straight.

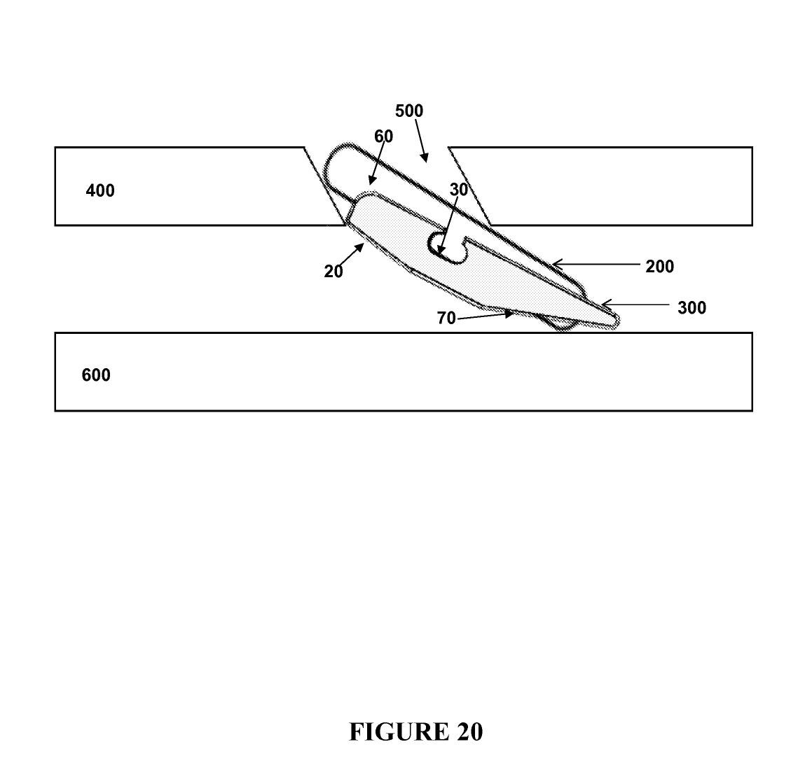

Embodiments herein describe an anchor. As used in this document, the term “anchor” means any device which may be used for attaching a tissue to or within a bone, including, but not limited to, a cortical button. In some embodiments, the anchor may be used in connection with a cortical bone or a cancellous bone. In some embodiments, the anchor may be used in connection with any type of surgery involving grafting tissue to bone. In some embodiments, the anchor may be used in connection with rotator cuff surgery (FIGS. 20A & 20B), proximal bicep repairs, distal bicep repair (FIG. 20C), anterior cruciate ligament (ACL) surgery, or the like.

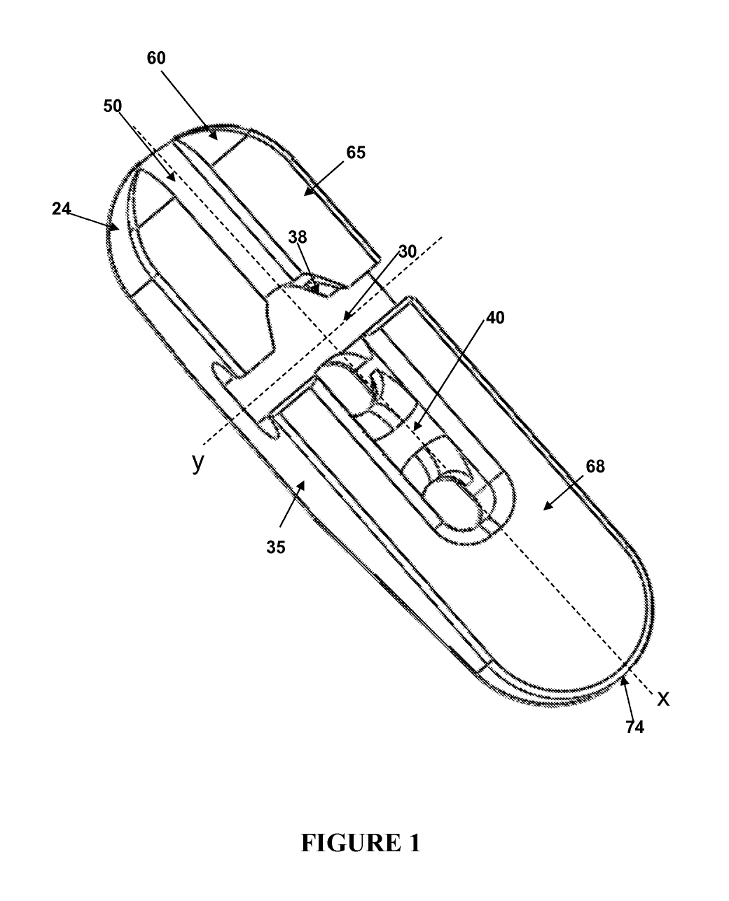

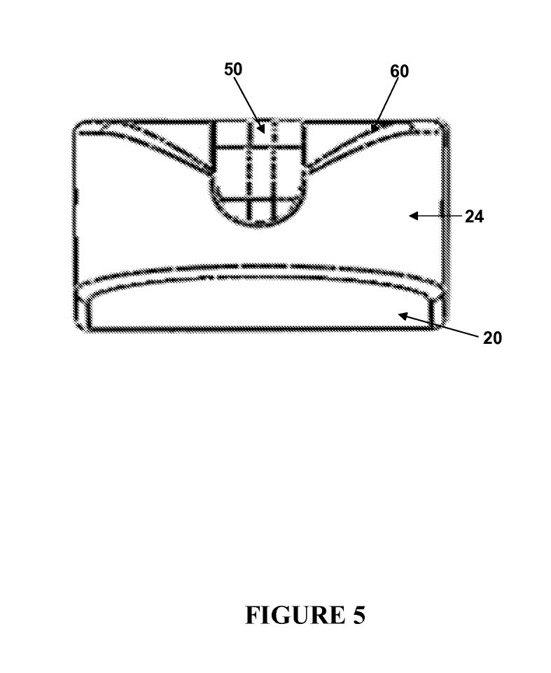

Referring to FIGS. 1-5, embodiments herein may be directed to an anchor comprising an angled bottom rear face 20 and an angled bottom leading face 70 which can be guided through both cortices of a bone and flipped on the outside of the bone. Each of the two angled bottom faces 20, 70 extends upward and away from a central face 45. In some embodiments, the anchor comprises a suture spool 40 which may be situated under the central face 45 and/or bottom leading face 70 and away from the center of the anchor's longitudinal axis. In other embodiments, an anchor and insertion tool is provided, wherein the anchor has a bottom rear face 20, a bottom leading face 70, and a tool receptor 30. In some embodiments, the tool receptor 30 may be a socket, slot, hole, hook or other receptor. In some embodiments, referring to FIGS. 1 and 5, the anchor may further include an angled top rear face 60. The top rear face 60 is positioned opposite the bottom rear face 20 and extends downward from a first top face portion 65. The anchor also may have a second top face portion 68 partially or fully separated from the first top face portion 65 by the tool receptor 30. The face orientations of the first top face portion 65, second top face portion 68 and central bottom face portion 45 may be substantially parallel to each other.

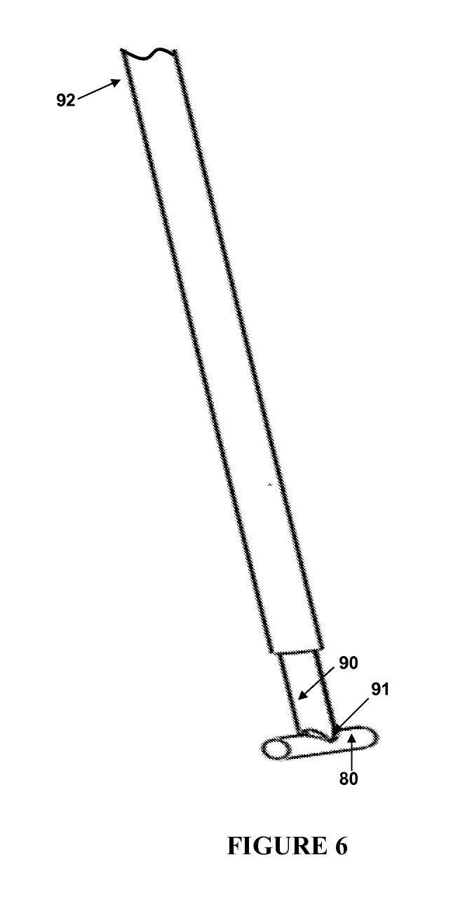

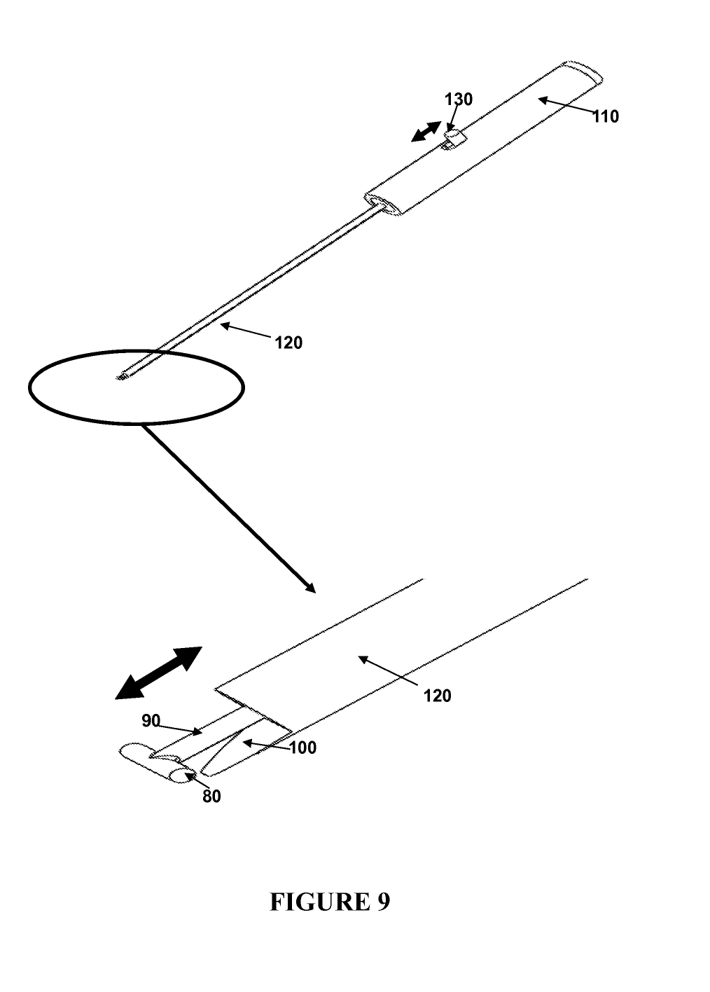

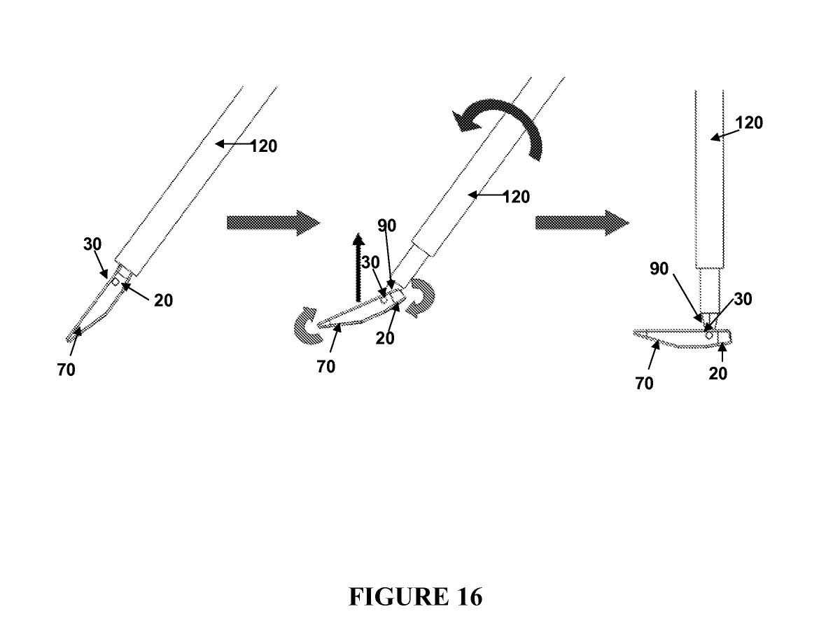

Referring to FIGS. 6 and 9, the insertion tool may have a rod member 90 and a tip 80 at a distal end which engages the tool receptor 30. In some embodiments, the tip 80 of the insertion tool is received by the tool receptor and is capable of actively pivoting the anchor causing the anchor to flip (FIG. 16). In some embodiments, the tip may allow the pivot structure to be positioned in space or positioned beyond a desired fixation point before rotation. In some embodiments, the tip 80 is complementary to the tool receptor 30.

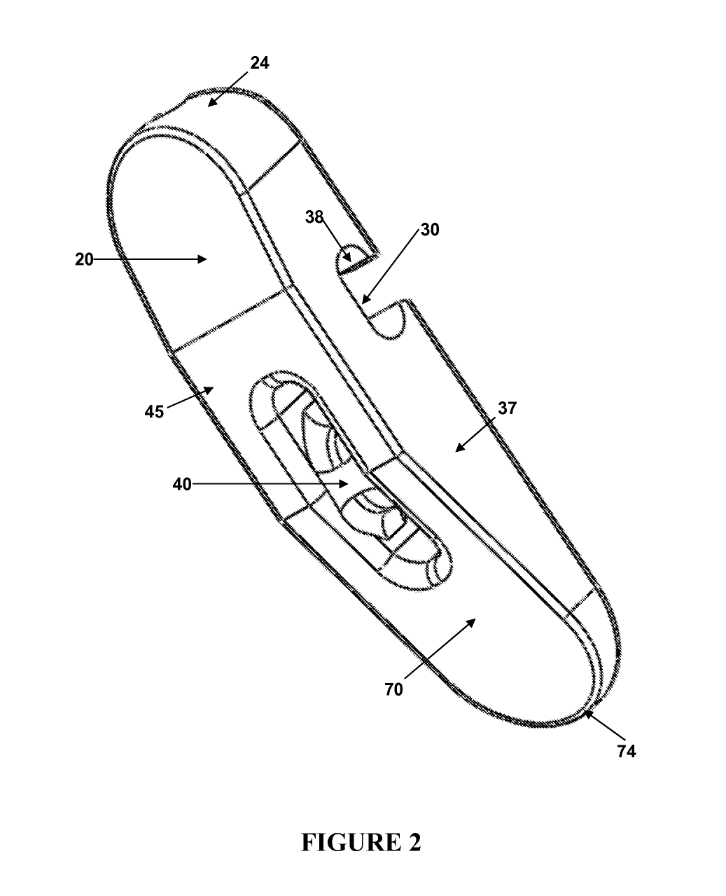

Referring to FIGS. 1 and 2, a curved leading side 74 is positioned between the second top face portion 68 and the bottom leading face 70. Similarly, a curved rear side 24 is positioned between the bottom rear face 20 and the first top face portion 65. The curved sides define a curved edge for their corresponding top and bottom face portions. In some embodiments, the sides 35, 37 of the anchor may be tapered inward from the bottom faces or the top faces towards the x axis (FIG. 1). In other embodiments, the sides of the anchor are straight. In some embodiments, the edges of the anchor may be curved. In some embodiments, the edges of the anchor may be curved angles. In other embodiments, the edges of the anchor may be hard angles.

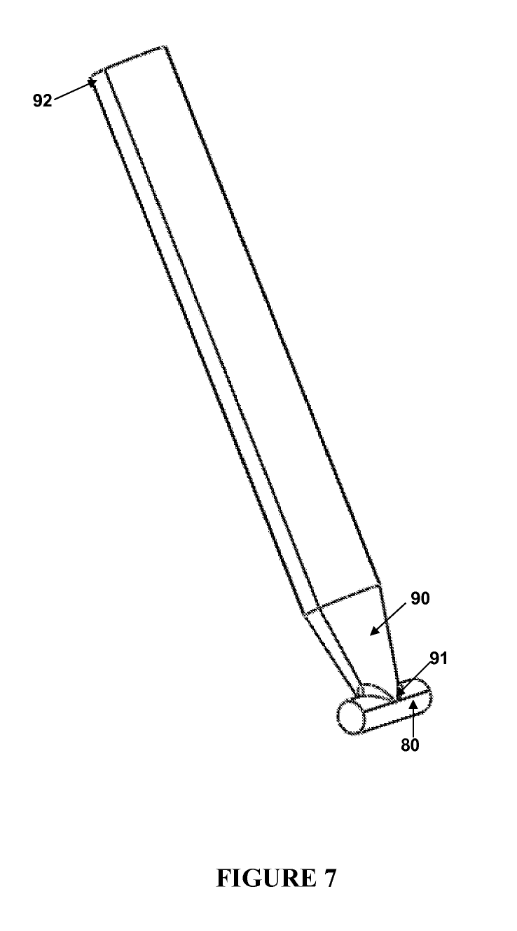

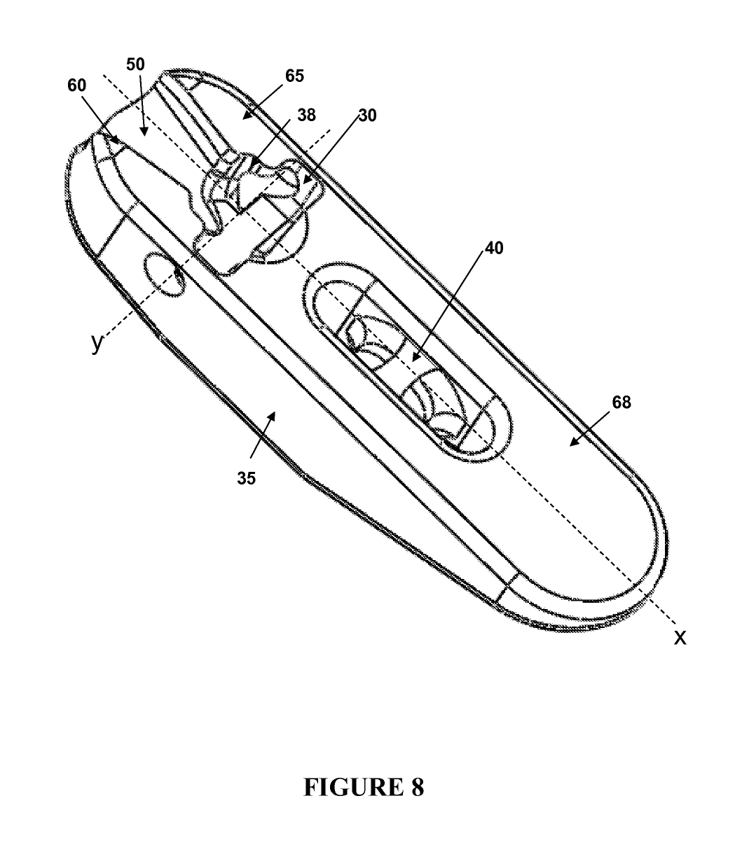

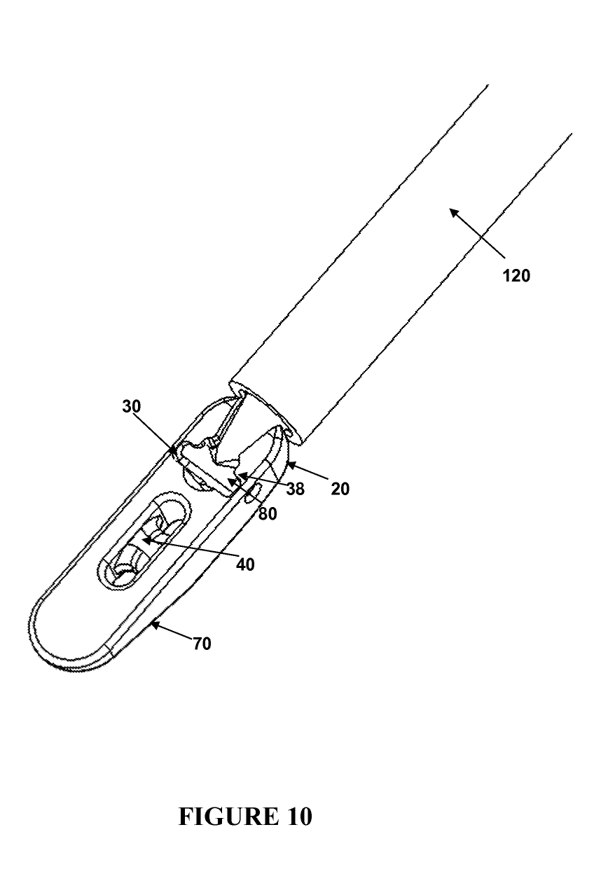

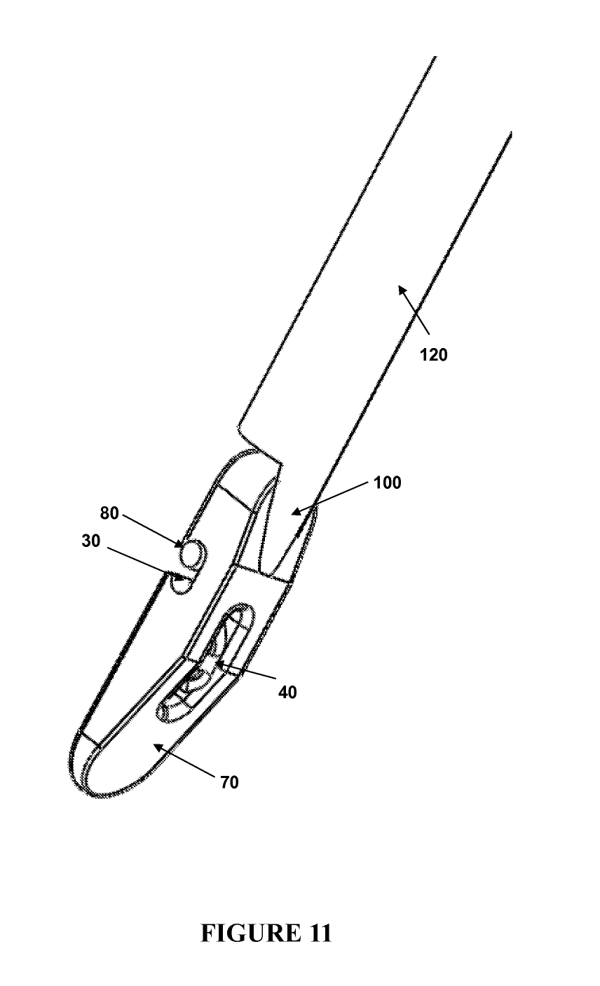

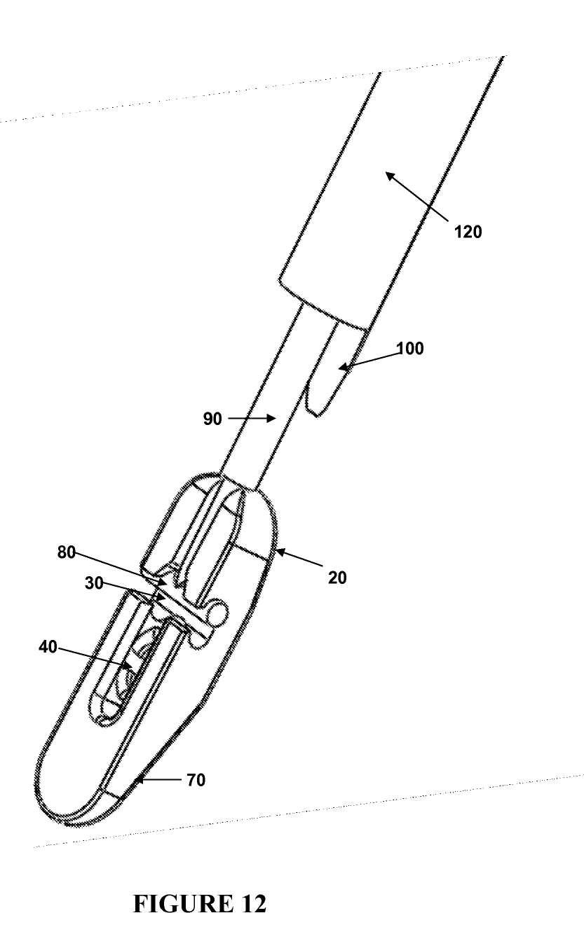

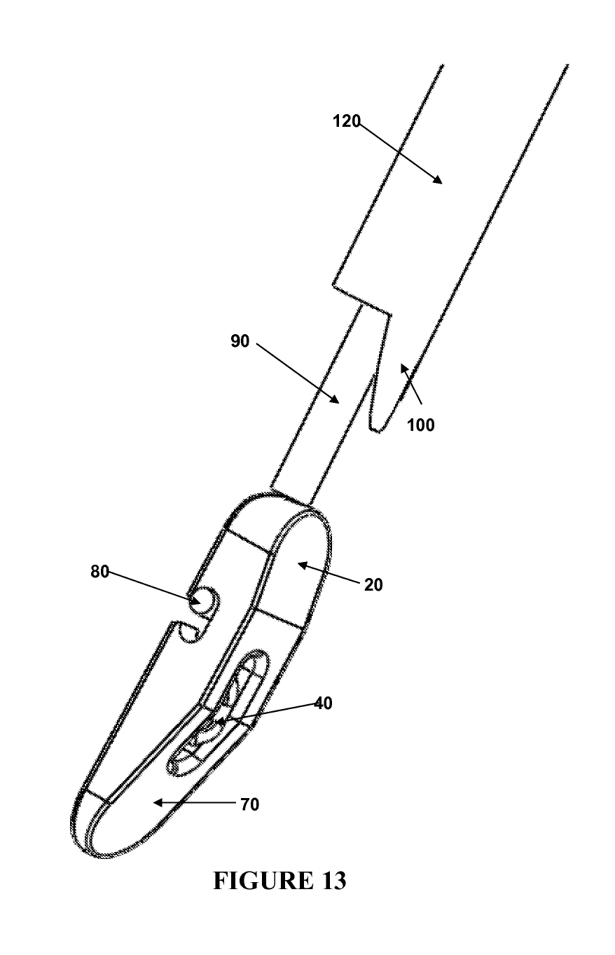

Embodiments herein may also be directed to an insertion tool for use with an anchor of embodiments herein. Referring to FIGS. 6 and 9, in some embodiments, the insertion tool comprises a rod member 90 having a proximal end 92 and a distal end 91; and a tip 80 connected to the rod member 90 on the distal end 91, wherein the tip 80 is shaped for mating with a tool receptor 30 in the anchor. In some embodiments, the tip 80 may be any shape which can suitably engage the tool receptor 30 of the anchor. In some embodiments, the tip 80 may be any shape which is complementary to the tool receptor 30 of the anchor. In some embodiments, the tip 80 may be a T-shaped tip, a pointed tip, a ball tip, an L-shaped tip, or the like. For example, in an embodiment where the tip 80 is a ball tip, the tip 80 inserts into the tool receptor 30 which can be complementarily shaped like a socket, thus allowing the tool and anchor to interact in a ball and socket motion. In some embodiments, referring to FIG. 7, the rod member 90 may be tapered towards distal end 91. In some embodiments, referring to FIG. 8, the anchor may include a tool receptor 30 and groove 50 shaped to receive a tapered rod member 90 as shown in FIG. 7. In some embodiments, referring to FIGS. 10-14, the insertion tool further includes a cannulated covering 120 from which the rod member 90 can extend or retract. In some embodiments, the rod member 90 may be at least partially enclosed within the cannulated covering 120 while the anchor-insertion tool assembly is guided into the hole in a bone. In some embodiments, the cannulated covering 120 may further include a lip 100. As shown in FIG. 11, the lip 100 may engage a portion of the anchor, for example, without limitations, the bottom rear face 20, and prevent the anchor from flipping while anchor is being guided into the bone canal. In some embodiments, as seen in FIGS. 12-13, the insertion tool may further include an actuator 130 for extending the rod member 90 from the cannulated covering 120. In some embodiments, as shown in FIG. 16, extending the rod member 90 allows the tip 80 to pivot in the tool receptor 30, thereby flipping the anchor. In some embodiments, when the rod member 90 extends the lip 100 is disengaged. In some embodiments, the actuator 130 may comprise a release, a spring, a plunger, a slide or a combination thereof. In some embodiments, the insertion tool may further include a lock for preventing the anchor from flipping until the lock is released. The lock prevents the anchor from accidentally deploying.

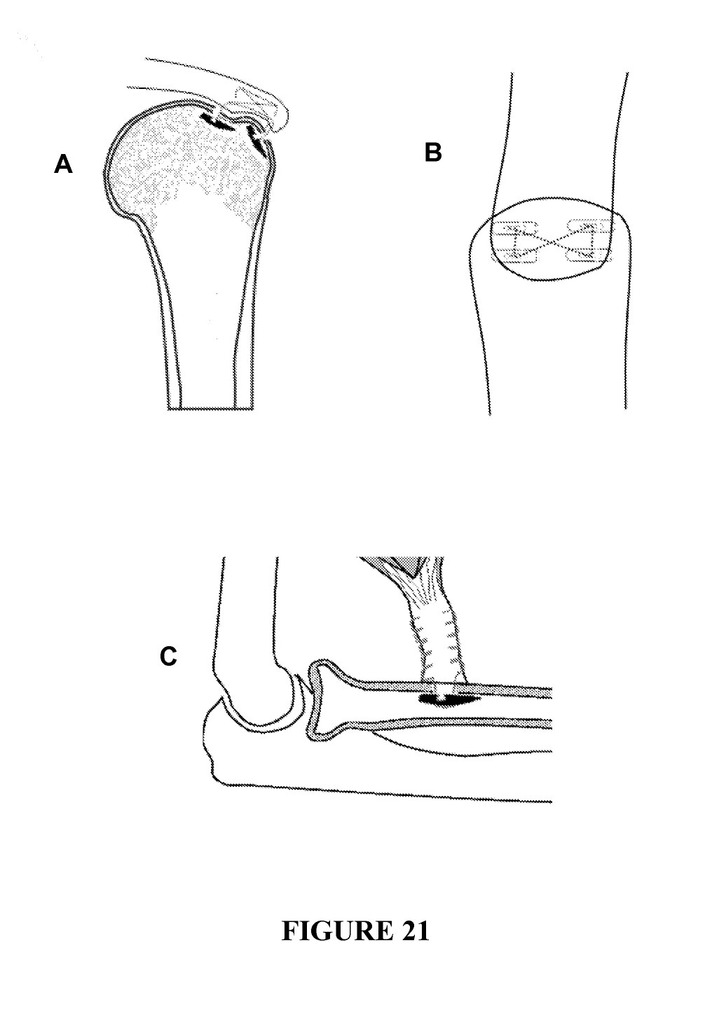

In further embodiments, without wishing to be bound by theory, the inventors have found it desirable to insert and flip the anchor over the far side of the near cortex when it is placed within the intramedullary canal of the diaphysis of a bone or within the cancellous bone of the metaphyseal or epiphyseal regions of a bone. This would only require drilling through one of the cortices which may help reduce the risk of injury from drilling. However, due to the limited space available inside a bone, properly flipping and securing the button can be difficult with currently available button and tool designs. Accordingly, embodiments herein may be directed to an anchor designed to be flipped in the intramedullary canal or within cancellous bone. In some embodiments, an inserter tool and anchor may be designed to flip the anchor in the intramedullary canal or within cancellous bone (FIG. 21). As shown in FIG. 21, in some embodiments, the anchor may be used during a double row rotator cuff repair, wherein the anchor may be flipped within the cancellous region of bone in the humerus (FIGS. 21A and 21B). In other embodiments, the anchor may be used for a distal biceps tendon repair, wherein the anchor may be flipped within the intramedullary canal of the radius (FIG. 21C).

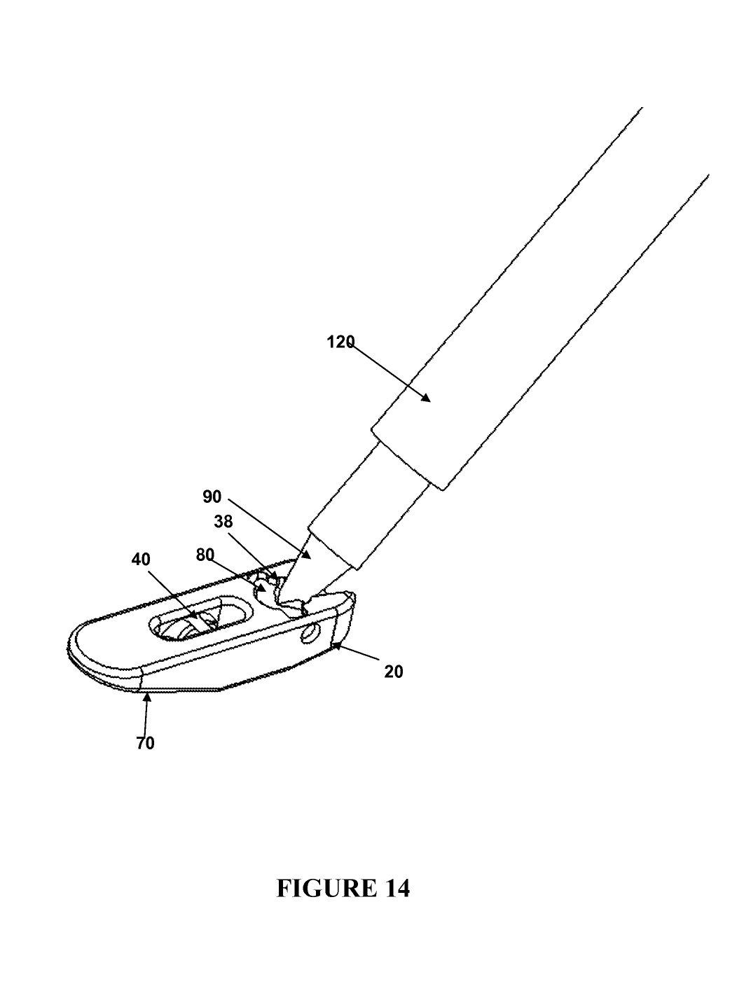

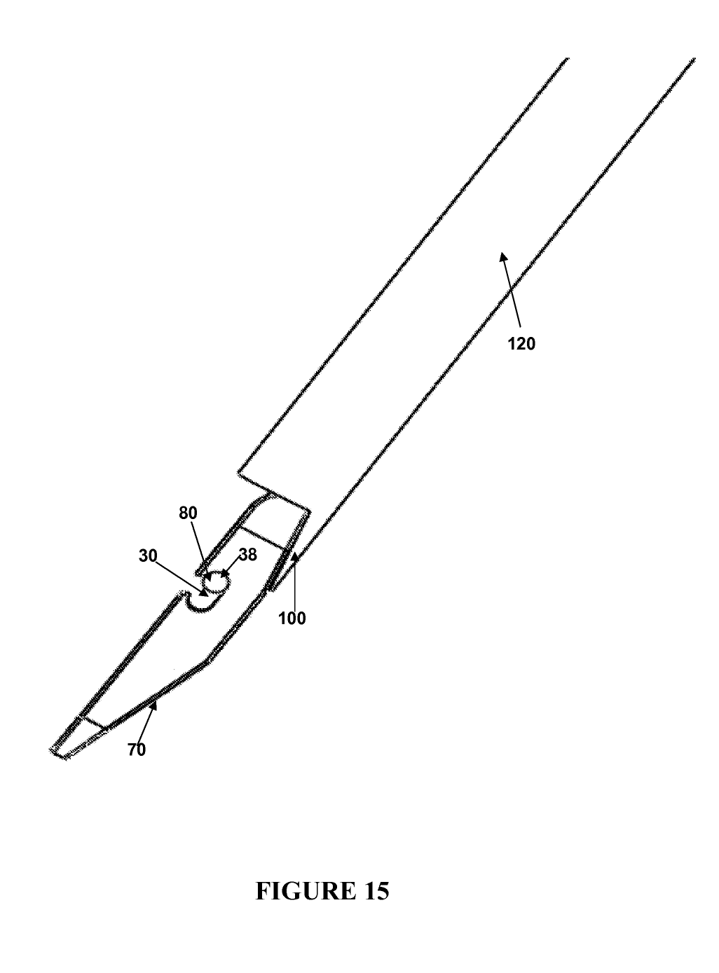

Thus, in some embodiments, using FIGS. 14-16 as examples, the anchor is movable relative to the insertion tool between a first position (FIG. 15) wherein the longitudinal axes of the insertion tool and the anchor are generally parallel with a portion of the insertion tool adjacent the tip 80 residing within the tool receptor 30 and a second position (FIG. 14) wherein the longitudinal axes of the insertion tool and the anchor move to a generally perpendicular position (i.e. the anchor is “flipped”). Referring to FIG. 16, for example, an exemplary anchor and insertion assembly may move from the first position on the far left to an intermediate flipping position in the middle to a flipped second position on the far right. In some embodiments, referring to FIGS. 1 and 2 as an example, an anchor comprises an angled bottom leading face 70 and an angled bottom rear face 20. The interfaces between the bottom center face 45 and either or both of the bottom rear face 20 need not be sharp and the angles may be curved. In such embodiments, the angled bottom leading face 70 and/or the angled bottom rear face 20 may form a curved bottom. In some embodiments, the angled bottom leading face 70 and the central bottom face 45 form an angle in the range from about 1 degrees to about 45 degrees, from about 1 degrees to about 30 degrees, from about 1 degrees to about 25 degrees, from about 1 degrees to about 20 degrees, from about 1 degrees to about 15 degrees, from about 5 degrees to about 45 degrees, from about 5 degrees to about 30 degrees, from about 5 degrees to about 25 degrees, from about 5 degrees to about 20 degrees, from about 5 degrees to about 15 degrees. In some embodiments, the angled bottom rear face 20 and the central bottom face 45 form an angle in the range from about 1 degrees to about 45 degrees, from about 1 degrees to about 30 degrees, from about 1 degrees to about 25 degrees, from about 1 degrees to about 20 degrees, from about 1 degrees to about 15 degrees, from about 5 degrees to about 45 degrees, from about 5 degrees to about 30 degrees, from about 5 degrees to about 25 degrees, from about 5 degrees to about 20 degrees, from about 5 degrees to about 15 degrees. In some embodiments, the central bottom face 45 and angled bottom rear face 20 and/or the angled bottom leading face 70 may together form a fully or partially curved bottom.

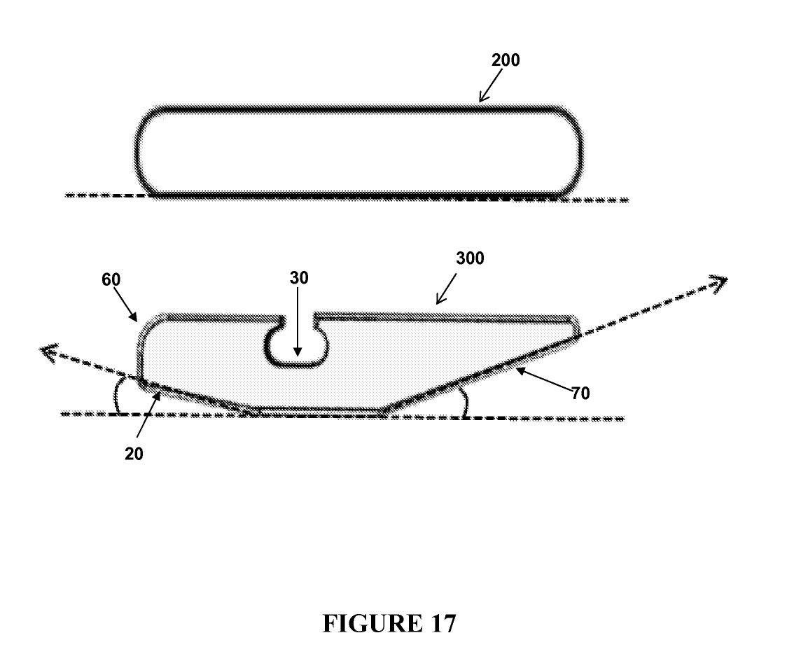

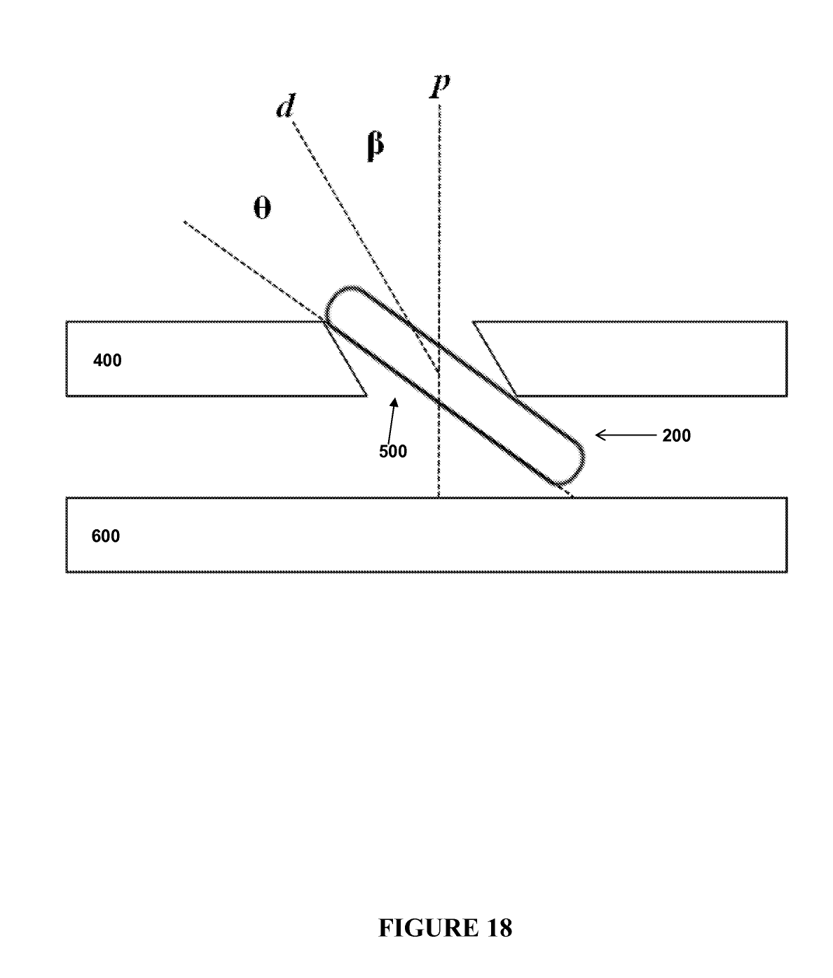

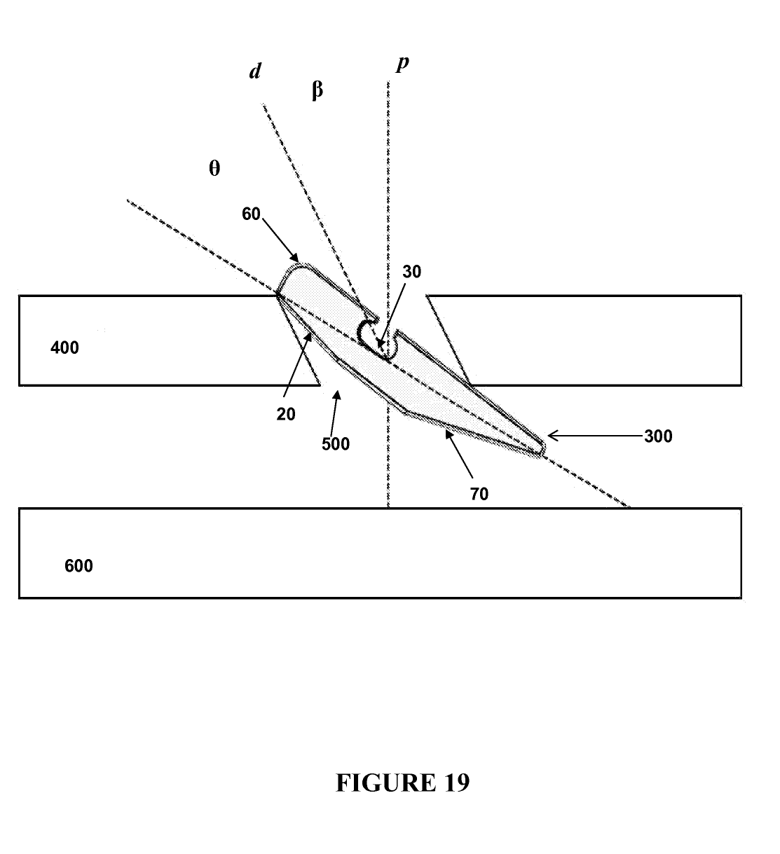

Without wishing to be bound by theory, referring to FIGS. 17-20, it is believed that the configuration (angle or curve) of the bottom leading face 70 may allow the anchor to slide further into the intramedullary canal before contacting the near side of the far cortex 600. As shown in FIG. 17, an anchor 300 encompassing an embodiment herein has an angled bottom leading face 70 and an angled bottom rear face 20 that is not present in an anchor 200 in the prior art which have a generally rectangular shaped cross-section. FIG. 18 shows the limitations of trying to implant an anchor 200 having a generally rectangular shaped cross-section into the intramedullary canal. The limited space in the intramedullary canal does not allow for proper insertion or flipping of the anchor 200. In contrast, FIG. 19 illustrates the approach angle of an anchor 300 of an embodiment herein. Once the anchor 300 contacts the near side of the far cortex 600, the angle of the bottom leading face 70 may promote flipping of the anchor 300 by allowing the anchor 300 to slide along the near side of the far cortex 600. As can be seen from FIGS. 19 and 20, once the anchor enters the intramedullary canal, the angled top rear edge 60 may aid in flipping and securing the anchor 300 against the far side of the near cortex 400 and disengaging the insertion tool from the anchor 300 so that the anchor 300 covers the drilled hole 500.

In some embodiments, the shape of the anchor 300 may improve the probability of the anchor 300 correctly flipping inside the intramedullary canal of the bone. Furthermore, in some embodiments, use of the insertion tool in combination with the anchor may further improve the probability of the anchor correctly flipping inside the intramedullary canal of a cortical bone.

Referring to FIG. 19, in some embodiments, to use the anchor a hole 500 may be drilled into the near cortex 400 of the diaphysis of a bone or within the cancellous bone of the metaphyseal or epiphyseal regions of a bone. In some embodiments, the angle of the bottom leading face 70 and the bottom rear face 20 may be designed to allow for the anchor 300 to rotate to a shallower approach angle into the drilled hole 500. The approach angle of the anchor 300 may be adjusted by the surgeon according to methods known to the surgeon. The approach angle may depend on the characteristics of the subject being treated, e.g., bone size, drill angle, age, weight, health, types of concurrent treatment, if any, and frequency of treatments, and can be easily determined by one of skill in the art (e.g., by the surgeon). In addition, the approach angle may depend on the drill angle β and the amount that the anchor 300 is able to rotate inside the hole θ. In some embodiments, the anchor 300 may be capable of rotating slightly during approach (FIG. 19). This additional rotation of the anchor may be accomplished because of the angle of the bottom rear face 20. The additional rotation may allow the anchor 300 to slide further at a shallow angle into the intramedullary canal before contacting the near side of the far cortex 600. In some embodiments, the additional rotation of the anchor θ may be from about 5 to about 30 degrees from the drill angle line d (with 0 degrees meaning in line with the center axis of the drill hole, where the anchor is placed in the hole at the drill angle, β). In some embodiments, the additional rotation of the anchor θ may be from about 5 to about 25 degrees, from about 5 to about 20 degrees, from about 5 to about 15 degrees, from about 5 to about 10 degrees, from about 10 to about 25 degrees, from about 10 to about 20 degrees, or from about 5 to about 15 degrees from a drill angle line d. FIG. 20 illustrates a comparison of the angle of entry into the intramedullary canal of an anchor 200 with a generally rectangular shaped cross-section; and an anchor 300 according to an embodiment described herein.

In some embodiments, a hole 500 may be drilled into the bone at an angle β of from about 0 degrees to about 45 degrees from a line p perpendicular to the bone surface. In some embodiments, the drill angle β may be from about 0 degrees to about 40 degrees, from about 0 degrees to about 35 degrees, from about 0 degrees to about 30 degrees, from about 0 degrees to about 25 degrees, from about 0 degrees to about 20 degrees, from about 0 degrees to about 15 degrees, from about 0 degrees to about 10 degrees, or from about 0 degrees to about 5 degrees from a line p perpendicular to the bone surface.

In some embodiments, the anchor of embodiments herein may be inserted into a hole in a bone at an approach angle (θ+β) of from about 5 to about 75 degrees, from about 5 to about 70 degrees, from about 5 to about 65 degrees, from about 5 to about 60 degrees, from about 5 to about 55 degrees, from about 5 to about 50 degrees, from about 5 to about 45 degrees, from about 5 to about 40 degrees, from about 5 to about 35 degrees, from about 5 to about 30 degrees, from about 5 to about 20 degrees, from about 5 to about 15 degrees, from about 5 to about 10 degrees, from about 10 to about 75 degrees, from about 10 to about 65 degrees, from about 10 to about 55 degrees, from about 10 to about 45 degrees, from about 10 to about 35 degrees, from about 10 to about 25 degrees, from about 5 to about 20 degrees, from about 10 to about 15 degrees from a line p perpendicular to the bone surface.

In some embodiments, the anchor may be any size suitable for implantation in a bone, including, without limitation, bones of the shoulder, elbow, hand, wrist, knee or foot. In some embodiments, the anchor may be sized for near cortex anchor fixation. In some embodiments, the anchor may be from about 5 mm to about 30 mm in length, from about 5 mm to about 25 mm, from about 5 mm to about 20 mm, from about 5 mm to about 15 mm, from about 10 mm to about 20 mm, or from about 10 mm to about 15 mm.

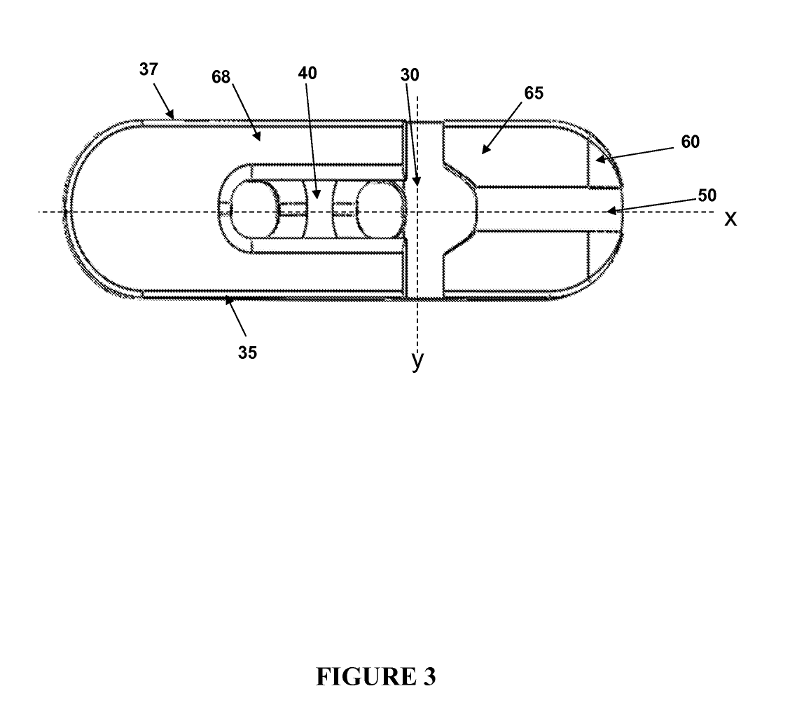

In some embodiments, the anchor may further comprise a suture spool 40. In some embodiments, the suture spool 40 may be situated away from the center of the longitudinal axis (x) of the anchor (FIGS. 1 and 3). In some embodiments, the suture spool 40 may be situated away from the center of the longitudinal axis by about 0.01 to about 10 mm. In some embodiments, the suture spool 40 may be situated away from the center of the longitudinal axis by about 0.01 to about 5 mm. In some embodiments, the suture spool 40 may be situated away from the center of the longitudinal axis by about 0.01 to about 3 mm. In some embodiments, the suture spool 40 may be situated away from the center of the longitudinal axis by about 0.01 to about 2 mm. In some embodiments, the suture spool 40 may be situated away from the center of the longitudinal axis by about 0.01 to about 1 mm. In some embodiments, the suture spool 40 may be situated away from the center of the longitudinal axis by about 0.01 to about 0.5 mm. Without wishing to be bound by theory, it is believed that the suture spool 40 may help create an off-center moment or leverage about an axis (y) generally perpendicular to the longitudinal axis of the anchor when sutures are pulled so that the anchor may be flipped to the second position easily.

In some embodiments, the anchor further includes a tool receptor 30 (FIGS. 1-5). The tool receptor 30 may allow for an insertion tool to engage the anchor. In some embodiments, the tool receptor 30 may be a pivot point to aid in flipping the anchor. In some embodiments, the tip may allow the pivot structure to be positioned in space or positioned beyond a desired fixation point before rotation. In some embodiments, the insertion tool engages the tool receptor 30 and actively pivots the anchor causing the anchor to flip. In some embodiments, the tool receptor may be any shape which allows an insertion tool to engage the anchor. In some embodiments, the tool receptor may be a shape that is complementary to a tip 80 on the distal end of the insertion tool. In some embodiments, the tool receptor 30 may be any socket shape. In some embodiments, the tool receptor 30 may be T-shaped, a pointed hole, a slot, a ball socket, L-shaped, or the like. In some embodiments, the tool receptor 30 may be shaped to allow insertion tool to be removed easily. In some embodiments, the tool receptor 30 may be shaped to allow insertion tool to be removed easily after anchor has been flipped, while still mating with anchor during flipping. In some embodiments, the tool receptor 30 may have walls that are angled past perpendicular or curved to allow the insertion tool to lock into the tool receptor while the anchor-insertion tool assembly is being guided into the bone and to aid in flipping the anchor. This may prevent the anchor from disengaging from the insertion tool. In other embodiments, the insertion tool may further comprise a lock to prevent the anchor from disengaging from the anchor-insertion tool assembly. In some embodiments, the insertion tool may further comprise a lock which has a complementary structure in the anchor to prevent the anchor from disengaging from the anchor-insertion tool assembly.

Surgically, it may be advantageous to minimize the size of the drill hole and the amount of material removed to form the hole. In some embodiments, the tool receptor 30 within the anchor body engages the pivot structure of the tool tip 80 such that the entire anchor/tool assembly may be no wider than the width of the anchor. For example, referring to FIG. 10, before being guided into the bone, a T-shaped tip 80 of the insertion tool may engage the anchor at tool receptor 30, which is shaped like an elongated slot to be complementarily to the T-shaped tip. In some embodiments, the curved walls of the tool receptor 30, the groove 50 and the off-center suture spool 40 of the anchor work in tandem to ensure that the anchor stays parallel to the insertion tool while being inserted into the bone and that the pivot axis of the anchor is positioned in space.

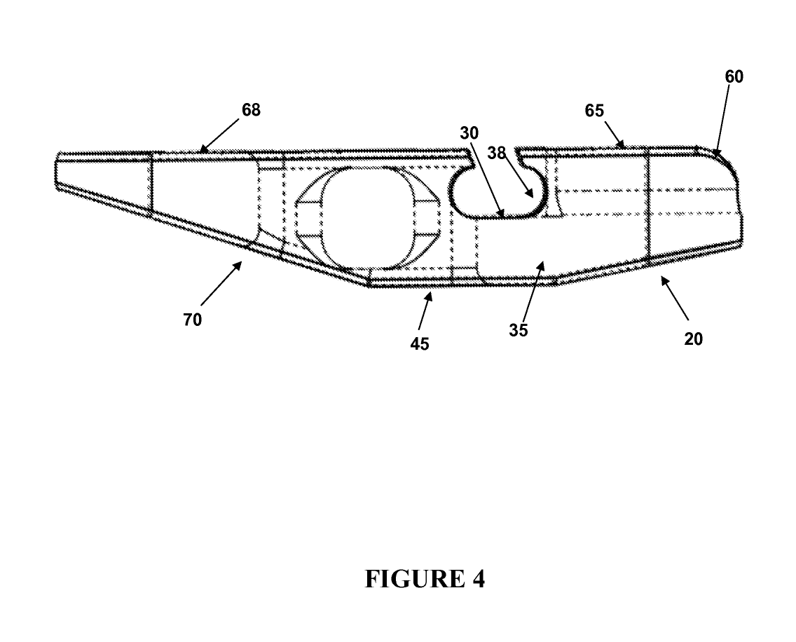

While being guided into the bone, the anchor may be aligned parallel to the insertion tool by a groove 50 in the anchor which forms a notch that fits at least a portion of the circumference of rod member 90 and by the walls of the tool receptor 30 that are curved to engage tip 80 of the insertion tool (FIGS. 4, 10, and 12). The groove 50 forms a notch which constrains the rod member 90 and tip 80 to provide alignment of the pivot axis (y-axis) within the insertion tool during the insertion of the anchor. In some embodiments, referring to FIGS. 7 and 8, the rod member 90 may be tapered and fit into a complementary groove 50 in the anchor.

In some embodiments, to prevent the anchor from disengaging from the pivot structure of the insertion tool during pivoting of the anchor, the curved back wall 38 of the tool receptor 30 may form a ledge which constrains the tip 80 to engage the back wall 38 of the tool receptor 30 (FIGS. 14-15). As shown in FIG. 8, the back wall 38 may be shaped so that it fits tip 80 of the insertion tool. In some embodiments, the tip 80 may actively pivot the anchor by engaging the back wall 38 of the tool receptor 30, which acts as a pivot point to move the anchor into the second position (FIG. 14). The curved back wall 38 may prevent disengagement of the anchor during insertion. Once the anchor has been inserted into the bone and flipped to cover the drilled hole, the insertion tool may be disengaged from the anchor by sliding the tip 80 forward so that it disengages from the back wall 38 and lifting the tip 80 out of tool receptor 30.

Once the anchor is in second position perpendicular to the insertion tool, the tip 80 of the insertion tool may disengage through the top opening of the ledge of the tool receptor 30 of the anchor and the insertion tool may be withdrawn. The tool receptor 30 and groove 50 allow the insertion tool to be removed from the top side of the anchor through the near cortex drill hole without contacting the edges of the drill hole, which could possibly inhibit or obstruct the insertion or pivoting of the anchor and which may necessitate a larger drill hole. Additionally, tool receptor 30 and groove 50 allow the insertion tool to be removed from the top side of the anchor through the near cortex drill hole without any horizontal translation (such as, for example, unclamping a tool from the outer width of the button or other horizontal separation of the tool tip from the tool receptor) of the tip to disengage the anchor. The tool receptor 30 and groove 50 allow the anchor to be parallel to the insertion tool as it is guided into the hole so that the size of the drill hole and the amount of material removed to form the hole may be minimized.

As used in the present disclosure, “actively pivots” refers to the anchor pivoting around the insertion tool's tip without the need for a separate biasing member. In some embodiments, the tip 80 of the insertion tool actively pivots the anchor by engaging the anchor at the tool receptor 30 and using the tool receptor 30 as a pivot point. In some embodiments, the tip 80 of the insertion tool actively pivots the anchor by working in tandem with the curved walls of the tool receptor 30, the groove 50 and the off-center suture spool 40 of the anchor.

In some embodiments, the anchor may be manufactured by computer numerically controlled (CNC) milling, CNC machining, or the like. In some embodiments, the anchor may be comprised of titanium, titanium alloy, stainless steel, chrome, nitinol, plastic, bioresorbable material, composite material, cobalt chrome alloy or any material that is suitable for implantation in the body. In some embodiments, the insertion tool may be manufactured by CNC milling, CNC machining, welding, stamping, forming or the like. In some embodiments, the insertion tool may be comprised of titanium, titanium alloy, stainless steel, chrome, nitinol, plastic, bioresorbable material, composite material, cobalt chrome alloy or the like. In some embodiments, the tip may be manufactured separately from the rod member and then welded, stamped, glued or otherwise adhered together. In other embodiments, the rod member and tip may be manufactured as one structure. In some embodiments, the insertion tool may also include a handle made of wood, metal, plastic or the like. In some embodiments, the insertion tool may also include an actuator 130 made of titanium, titanium alloy, stainless steel, chrome, nitinol, rubber, plastic, bioresorbable material, composite material, cobalt chrome alloy or the like. In some embodiments, the insertion tool may also include a cannulated covering 120 made of titanium, titanium alloy, stainless steel, chrome, nitinol, plastic, bioresorbable material, composite material, cobalt chrome alloy or the like. In some embodiments, the insertion tool may also include a lip made of titanium, titanium alloy, stainless steel, chrome, nitinol, plastic, bioresorbable material, composite material, cobalt chrome alloy or the like.

Embodiments herein also describe a method of using the anchor and insertion tool of embodiments described herein. Referring to FIGS. 10-13, in some embodiments, a method of using an anchor and an insertion tool comprises loading the anchor having a bottom rear face 20 and a bottom leading face 70 onto the insertion tool having a rod member 90 and tip 80 such that a longitudinal axis of the anchor is parallel to a longitudinal axis of the insertion tool, wherein a tip 80 of the insertion tool engages a tool receptor 30 of the anchor; guiding the anchor and insertion tool assembly into a drilled hole in a bone, pivoting the anchor such that the longitudinal axis of the anchor covers an opening of the drilled hole (FIGS. 16 and 19). In some embodiments, pivoting comprises actively pivoting the anchor using the tip 80 of the insertion tool in the tool receptor 30 (FIG. 16). In some embodiments, the anchor is guided through a hole in both cortices of a bone and flipped on the outside of the bone. In some embodiments, the anchor is guided through a hole in one cortex of the bone and flipped when the anchor reaches the far cortex of the intramedullary canal. In some embodiments, when flipped, the anchor covers an opening of the hole.

This invention and embodiments illustrating the method and materials used may be further understood by reference to the following non-limiting examples.

### Example 1

An anchor may be made by CNC milling titanium alloy which has a length of about 11 mm and a width of about 3 mm. The anchor may have a bottom leading edge having an angle of 15 degrees, as measured from the bottom edge. The anchor may have a bottom rear edge having an angle of 10 degrees, as measured from the bottom edge. The anchor may also have a T shaped tool receptor and a suture spool which is 0.5 mm off center on the longitudinal axis of the anchor.

### Example 2

An insertion tool may be made using CNC milling stainless steel including a rod member which has a length of about 10 inches and a diameter of about 1 mm, and a tip at the distal end of the rod member which is T-shaped to correspond with the tool receptor in the anchor of Example 1. The tip has a length of about 3.5 mm and a diameter of about 0.7 mm. The insertion tool may also have a cannulated covering made of stainless steel. The cannulated covering may also include a lip made of stainless steel to prevent the anchor from flipping as it is being guided into a hole through the bone. The lip may extend out from a portion of the cannulated covering and may engage the bottom rear edge of the anchor. The insertion tool may also have a handle made of plastic. The insertion tool also may have an actuator made of plastic. The actuator may be a spring loaded button which when pressed extends the rod member from the cannulated covering. The walls of the tool receptor of the anchor may be curved so as to lock onto the tip of the insertion tool while the anchor is in the anchor-insertion tool assembly being guided into the bone and may aid in near cortex anchor fixation.

Although the present invention has been described in considerable detail with reference to certain preferred embodiments thereof, other versions are possible. Therefore the spirit and scope of the invention should not be limited to the description and the preferred versions contained within this specification.

1. A cortical anchor and insertion tool assembly comprising:
an anchor including a tool receptor that receives a pivot structure;
an insertion tool including a rod member having a distal end, a proximal end, and a tip at the distal end of the rod member including the pivot structure which is configured to pivot the anchor at the tool receptor.

2. The assembly of claim 1, wherein the tip is configured to allow the pivot structure to be positioned in space or positioned beyond a desired fixation point before rotation.

3. The assembly of claim 1, wherein the anchor is about 5 mm to about 30 mm in length.

4. The assembly of claim 1, wherein the tool receptor comprises curved walls to engage the tip.

5. The assembly of claim 1, wherein the anchor comprises a suture spool to receive a suture.

6. The assembly of claim 5, wherein the suture spool is situated away from a center of a longitudinal axis of the anchor by from about 0.1 mm to about 5 mm.

7. The assembly of claim 1, wherein the anchor further comprises a bottom leading face and a bottom central face that together form an angle from about 5 to about 45 degrees.

8. The assembly of claim 7, wherein the anchor further comprises a bottom rear face that together with the bottom central face forms an angle from about 5 to about 45 degrees.

9. The assembly of claim 1, wherein the insertion tool further comprises a cannulated covering which at least partially covers the rod member.

10. The assembly of claim 9, wherein the cannulated covering comprises a lip to prevent the anchor from pivoting when inserted into an anchoring location.

11. The assembly of claim 9, wherein the insertion tool further comprises an actuator to extend the rod member from the cannulated covering.

12. The assembly of claim 10, wherein the insertion tool further comprises an actuator to extend the rod member from the cannulated covering past the lip.

13. A cortical anchor comprising:
a tool receptor configured to receive a pivot structure; and
a suture spool configured to receive a suture, wherein the spool is situated away from a center of a longitudinal axis of the anchor by from about 0.1 mm to about 5 mm.

14. The anchor of claim 13 further comprising a bottom leading face and a bottom central face that together form an angle from about 5 to about 45 degrees.

15. The anchor of claim 14 further comprising a bottom rear face that, together with the bottom central face, forms an angle from about 5 to about 45 degrees from a bottom edge of the anchor.

16. The anchor of claim 13, wherein the tool receptor comprises a T-shape, L-shape, a ball socket, a hole, or a combination thereof.

17. The anchor of claim 13, wherein the tool receptor includes curved walls.

18. A cortical anchor and insertion tool assembly comprising:
an anchor comprising:
a tool receptor configured to receive a pivot structure,
a central face, a leading face that, together with the central face, forms an angle from about 5 to about 45 degrees,
a rear face that, together with the central face, forms an angle from about 5 to about 45 degrees, and
a suture spool configured to receive a suture; and
an insertion tool including
a rod member, and
a tip having the pivot structure capable of actively pivoting the anchor at the tool receptor.

19. The assembly of claim 18, wherein the tip allows the pivot structure to be positioned in space or positioned beyond a desired fixation point before rotation.

20. The assembly of claim 18, wherein the suture spool is situated away from a center of a longitudinal axis of the anchor by from about 0.1 mm to about 5 mm.
